(19) United States
(12) Patent Application Publication
(10) Pub. No.: US 2018/0312900 A1
(43) Pub. Date: Nov. 1, 2018

(54) PROCESSES FOR INCREASING EXTRACTION OF ENZYMES FROM ANIMAL FEED AND MEASURING ACTIVITY OF THE SAME

(71) Applicant: AGRIVIDA, INC., Woburn, MA (US)

(72) Inventors: Xuemei LI, Belmont, MA (US); R. Michael RAAB, Arlington, MA (US)

(73) Assignee: AGRIVIDA, INC., Woburn, MA (US)

(21) Appl. No.: 15/569,583

(22) PCT Filed: May 20, 2016
(86) PCT No.: PCT/US2016/033472

§ 371 Date: Oct. 26, 2017

Related U.S. Application Data
(60) Provisional application No. 62/164,007, filed on May 20, 2015.

(51) Int. Cl. C12Q 1/42

(52) U.S. Cl. CPC C12Q 1/42; C12Y 301/03026

(57) ABSTRACT

Methods of increasing extraction of enzymes from animal feed and measuring enzyme activity are described herein.

### CROSS REFERENCE TO RELATED APPLICATIONS

This application claims the benefit of U.S. Provisional Application No. 62/164,007, which was filed May 20, 2015, and is incorporated herein by reference as if fully set forth.

### FIELD

The disclosure relates to a procedure for phytase extraction from feed samples and its activity assay. The activity assay detects the presence of inorganic phosphate, which is released from a sodium phytate substrate by hydrolytic enzymatic action of phytase.

### BACKGROUND

Phosphorus in grain-based animal diets is available in the form of phytic acid. The phosphate found in phytic acid can be digested by ruminant animals because the bacteria colonizing their guts produce phytase converting the non-digestible bound phosphate into the inorganic free form that can be easily absorbed. However, monogastric animals do not carry this type of bacteria, and thus, cannot utilize phytic acid as a source of phosphorus. For this reason, exogenous phytases are routinely added to diet formulations for feeding monogastric animals to improve their phosphate utilization.

Phytase (myo-inositol hexakisphosphate phosphorylase, EC 3.1.3.8) catalyzes the hydrolysis of phytic acid (myo-inositol hexakisphosphate), an indigestible, organic form of phosphorus, and releases a usable form of inorganic phosphorus. Traditionally, microbial phytases produced in fermentation are used for feed production either by spraying an enzyme on the surface of pelleted feed or adding them to feed in concentrated dry formulation. The production of microbial derived phytase in plants has cost advantages, and ground plant tissue can be mixed directly in the feed. Adding dry formulation or pulverized plant tissue to feed at a low inclusion rate creates new challenges for reliable measurement of the enzyme activity of the feed samples. An accurate assay is desirable for measuring phytase activity in feed formulations.

### SUMMARY

In an aspect, the invention relates to a method for measuring activity of phytase in an animal feed. The method includes mixing an amount of an animal feed with a carbonate-bicarbonate buffer to obtain a mixture. The animal feed includes phytase. The carbonate-bicarbonate buffer includes sodium carbonate at a concentration in a range from 10 mM to 500 mM and sodium bicarbonate at a concentration in a range from 10 mM to 500 mM. The method includes extracting the phytase from the mixture. The method also includes measuring the activity of an extracted phytase.

In an aspect, the invention relates to a method for extracting a feed enzyme. The method includes mixing an amount of animal feed with a carbonate-bicarbonate buffer to obtain a mixture. The animal feed includes a feed enzyme. The carbonate-bicarbonate buffer includes sodium carbonate at a concentration in a range from 10 mM to 500 mM and sodium bicarbonate at a concentration in a range from 10 mM to 500 mM. The method also includes extracting the feed enzyme from the mixture.

### BRIEF DESCRIPTION OF THE DRAWINGS

The following detailed description of the preferred embodiments of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 is a chart illustrating phytase activity at a temperature of 37° C. or 65° C. following extraction from feed using sodium acetate buffer, pH 5.5, 0.01% TWEEN 20® (diagonal striped bar), sodium borate buffer, pH 10, 0.01% TWEEN 20® (gray bar) or sodium carbonate/bicarbonate buffer, pH 10.8 (horizontally striped bar). Results for each buffer are reported for dilutions 5×, 10× and 20× and temperatures of 37° C. and 65° C.

FIG. 2 is a photograph of Western blot showing phytase extracted from feed using sodium carbonate/bicarbonate buffer, pH 10.8, sodium borate buffer, pH 10, and sodium acetate buffer, pH 5.5.

FIG. 3 is a chart illustrating phytase activity after extraction from feed samples using sodium carbonate/bicarbonate buffer at 22° C., 55° C., 65° C., 75° C., or 85° C. 50× dilution results are reported in the left bar for each temperature point panel. 100× dilution results are reported in the right bar for each temperature point panel.

FIG. 4 is a chart illustrating phytase activity recovered after extraction using sodium borate buffer, pH 10, 0.01% TWEEN 20® (bars 1 and 2) and sodium carbonate/bicarbonate buffer, pH 10.8 (bar 3).

FIGS. 5A-5B are charts illustrating percentage of the maximal activity recovered from 10 g feed formulated with 1000 FTU/kg (FIG. 5A) and 3000 FTU/kg (FIG. 5B) of phytase by using different volumes of sodium carbonate/bicarbonate extraction buffer.

FIG. 6 is a chart illustrating the phytase activity recovered from transgenic flour ground into three different particle sizes at two extractions using (1) sodium acetate buffer and (2) sodium carbonate/bicarbonate buffer.

FIG. 7 is a chart illustrating the phytase activity recovered from the feed formulated with 1000 FTU/kg and 3000 FTU/kg phytase using (1) sodium carbonate/bicarbonate buffer, (2) sodium carbonate/bicarbonate buffer with TWEEN 20®, and (3) sodium borate buffer with TWEEN 20®.

FIG. 8 is a chart illustrating stability of the phytase activity after storage in the (1) sodium carbonate/bicarbonate buffer, (2) sodium carbonate/bicarbonate buffer with TWEEN 20®, or (3) in the sodium borate buffer with TWEEN 20®.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

In an embodiment, a method for measuring activity of phytase in an animal feed is provided. The method may comprise mixing an amount of an animal feed with a carbonate-bicarbonate buffer to obtain a mixture. The animal feed may comprise phytase. The carbonate-bicarbonate buffer may include sodium carbonate and sodium bicarbonate. The method may comprise extracting the phytase from the mixture. The method may also comprise measuring the activity of an extracted phytase.

As used herein, “phytase” is an enzyme capable of catalyzing the hydrolysis of phytic acid. Prior to inclusion in the animal feed, the phytase may be produced in a genetically engineered host. The host may be, but is not limited to, a plant cell, a bacterial cell, a mammalian cell, or a yeast cell. The host may be the bacterial cell. The bacterial cell may be an Escherichia coli cell.

In an embodiment, the concentration of sodium carbonate may be in a range from 10 mM to 500 mM. The concentration of sodium bicarbonate may be in a range from 10 mM to 500 mM. Each of the foregoing concentration ranges may be subdivided. The concentration of each of the sodium carbonate and sodium bicarbonate may be subdivided between any two values chosen from 10 mM increments within the described range (endpoints inclusive). The concentration of any one reactant may be a specific value within its respective ranges. The concentration of sodium carbonate in the carbonate-bicarbonate buffer may be the same as the concentration of sodium bicarbonate. The concentration of sodium carbonate may be 30 mM. The concentration of sodium bicarbonate may be 30 mM. The concentration of sodium carbonate in buffer may differ from the concentration of sodium bicarbonate.

In an embodiment, a pH of the carbonate-bicarbonate buffer may be 10.00, or greater. A pH of the carbonate-bicarbonate buffer may be within a range from 10.00 to 14.00, endpoints inclusive. The pH of the carbonate-bicarbonate buffer may be 10.00, 10.5, 11.00, 11.5, 12.00, 12.5, 13.00, 13.5, or 14.00. The pH may be in a range between and including 10.0 and 11.0, 11.0 and 12.0, 12.0 and 13.0, 13.0 and 14.0. The pH may be any one integer value pH selected from those including and between 10.0 and 14.00. The pH may be any pH including and between 11.00 and 13.00. The pH may be 10.00.

In an embodiment, the carbonate-bicarbonate buffer may further comprise a nonionic detergent. As used herein, the nonionic detergent refers to detergents that do not produce ions in aqueous solution. The nonionic detergent may be a polysorbate-type nonionic detergent formed by the ethoxylation of sorbitan before the addition of lauric acid. The polysorbate-type nonionic detergent may be polyoxyethylene (20) sorbitan monolaurate. The polysorbate-type nonionic detergent may be TWEEN 20®. The TWEEN 20® may be included in the carbonate-bicarbonate buffer at a concentration in a range from 0.001% (v/v) to 1.0% (v/v). The concentration of TWEEN 20® may be subdivided between any two values chosen from 0.001% increments within the described range (endpoints inclusive). The concentration of any one TWEEN 20® may be a specific value within the described range.

In an embodiment, the amount of feed used in the method may be within a range from 100 g to 500 g, endpoints inclusive. The amount of feed may be 100 g, 150 g, 200 g, 250 g, 300 g, 350 g, 400 g, 450 g or 500 g. The amount of feed may be an amount from 100 g to 150 g, from 150 g to 200 g, from 200 g to 250 g, from 250 g to 300 g, from 300 g to 350 g, from 350 g to 400 g, from 400 to 450 g, or from 450 g to 500 g, endpoints inclusive. The amount of feed within any one of the ranges herein may be any value between any two of the points included in the range. The amount of feed may be greater than 500 g. As used herein, the term “animal feed” refers to any food, feed, feed composition, diet, preparation, additive, supplement, or mixture suitable and intended for intake by animals for their nourishment, maintenance, or growth.

In an embodiment, the method may further comprise grinding an animal feed to form flour prior to the step of mixing. The flour may comprise particles having a size within a range of 250 μm to 6,000 μm, endpoints inclusive. The flour may comprises particles having a size from 250 μm to 300 μm, from 300 μm to 400 μm, from 400 μm to 500 μm, from 500 μm to 600 μm, from 600 μm to 700 μm, from 700 μm to 800 μm, from 800 μm to 900 μm, from 900 μm to 1,000 μm, from 1,000 μm to 1,100 μm, from 1,100 μm to 1,200 μm, from 1,200 μm to 1,300 μm, from 1,300 μm to 1,400 μm, from 1,400 μm to 1,500 μm, from 1,500 μm to 1,600 μm, from 1,600 μm to 1,700 μm, from 1,700 μm to 1,800 μm, from 1,800 μm to 1,900 μm, from 1,900 μm to 2,000 μm, from 2,000 μm to 2,100 μm, from 2,100 μm to 2,200 μm, from 2,200 μm to 2,300 μm, from 2,300 μm to 2,400 μm, from 2,400 μm to 2,500 μm, from 2,500 μm to 2,600 μm, from 2,600 μm to 2,700 μm, from 2,700 μm to 2,800 μm, from 2,800 μm to 2,900 μm, from 2,900 μm to 3,000 μm, from 3,000 μm to 3,100 μm, from 3,100 μm to 3,200 μm, from 3,200 μm to 3,300 μm, from 3,300 μm to 3,400 μm, from 3,400 μm to 3,500 μm, from 3,500 μm to 3,600 μm, from 3,600 μm to 3,700 μm, from 3,700 μm to 3,800 μm, from 3,800 μm to 3,900 μm, from 3,900 μm to 4,000 μm, from 4,000 μm to 4,100 μm, from 4,100 μm to 4,200 μm, from 4,200 μm to 4,300 μm, from 4,300 μm to 4,400 μm, from 4,400 μm to 4,500 μm, from 4,500 μm to 4,600 μm, from 4,600 μm to 4,700 μm, from 4,700 μm to 4,800 μm, from 4,800 μm to 4,900 μm, from 4,900 μm to 5,000 μm, from 5,000 μm to 5,100 μm, from 5,100 μm to 5,200 μm, from 5,200 μm to 5,300 μm, from 5,300 μm to 5,400 μm, from 5,400 μm to 5,500 μm, from 5,500 μm to 5,600 μm, from 5,600 μm to 5,700 μm, from 5,700 μm to 5,800 μm, from 5,800 μm to 5,900 μm, from 5,900 μm to 6,000 μm, endpoints inclusive. The size of particles within any one of the ranges herein may be any value between any two of the size points included in the range. The size may be from 250 μm to 1000 μm, endpoints inclusive. The size may be at least 250 μm.

In an embodiment, an amount of animal feed may be an amount of flour produced after grinding the animal feed. The amount of flour used in the method may be within a range from 1 g to 500 g, endpoints inclusive. The amount of flour may be 1 g, 2 g, 3 g, 4 g, 5 g, 6 g, 7 g, 8 g, 9 g, 10 g, 20 g, 30 g, 40 g, 50 g, 60 g, 70 g, 80 g, 90 g, 100 g, 150 g, 200 g, 250 g, 300 g, 350 g, 400 g, 450 g or 500 g. The amount of flour may be an amount from 1 g to 10 g, from 10 g to 20 g, from 20 g to 30 g, from 30 g to 40 g, from 40 g to 50 g, from 50 g to 60 g, from 60 g to 70 g, from 70 g to 80 g, from 80 g to 90 g, from 90 g to 100 g, 100 g to 150 g, from 150 g to 200 g, from 200 g to 250 g, from 250 g to 300 g, from 300 g to 350 g, from 350 g to 400 g, from 400 to 450 g, or from 450 g to 500 g, endpoints inclusive. The amount of flour within any one of the ranges herein may be any value between any two of the points included in the range. The amount of flour may be less than 500 g.

In an embodiment, extracting phytase from the mixture may be performed by any extraction procedure known in the art. The extraction procedure may be a procedure described herein in Example 2, herein.

In an embodiment, a temperature of the mixture during the step of extracting may be in the range 20° C. to 80° C., endpoints inclusive. The temperature of the mixture may be 20° C., 25° C., 30° C., 35° C., 40° C., 45° C., 50° C., 55° C., 65° C., 70° C., 75° C., or 80° C., endpoints inclusive. The temperature of the mixture may be in the range 20° C. to 25° C., 20° C. to 30° C., 20° C. to 35° C., 20° C. to 40° C., 20° C. to 45° C., 20° C. to 50° C., 20° C. to 55° C., 20° C. to 60° C., 20° C. to 65° C., 20° C. to 70° C., 20° C. to 75° C., or less than 80° C. The temperature within any one of the ranges herein may be any value between any two of the temperature points included in the range. The temperature of the mixture may be 55° C.

In an embodiment, the amount of phytase extracted from feed using the carbonate-bicarbonate buffer at a temperature 37° C. may be greater than the amount of the phytase extracted by using the sodium borate buffer at the same temperature. The amount of phytase extracted from feed using the carbonate-bicarbonate buffer at a temperature 37° C. may be greater than the amount of the phytase extracted by using the sodium acetate buffer at the same temperature.

In an embodiment, the extracted phytase may be stored in a carbonate-bicarbonate buffer for a period of time. The extracted phytase may be stored for a period of time in a range from one hour to thirty days, endpoints inclusive. The time period may be 1 hour, 2 hours, 3 hours, 4 hours, 5 hours, 10 hours, 15 hours, 20 hours, 1 day, 2 days, 3 days, 4 days, 5 days, 10 days, 15 days, 20 days, 25 days, or 30 days. The time period may be any one integer value selected from those including and between value points, endpoints inclusive. The time period may be less than 30 days. The time period may be less than 20 days. The time period may be less than 10 days. The time period may be less than 1 day.

In an embodiment, the step of measuring the phytase activity of may be performed at a temperature within a range of 20° C.-80° C., endpoints inclusive. The temperature of the mixture may be 20° C., 25° C., 30° C., 35° C., 37° C., 40° C., 42° C., 45° C., 50° C., 55° C., 65° C., 70° C., 75° C., or 80° C., endpoints inclusive. The temperature of the mixture may be in the range 20° C. to 25° C., 20° C. to 30° C., 20° C. to 35° C., 20° C. to 37° C., 20° C. to 40° C., 20° C. to 42° C., 20° C. to 45° C., 20° C. to 50° C., 20° C. to 55° C., 20° C. to 60° C., 20° C. to 65° C., 20° C. to 70° C., 20° C. to 75° C., or less than 80° C. The temperature within any one of the ranges herein may be any value between any two of the temperature points included in the range. The temperature may be 37° C.

In an embodiment, a method for extracting a feed enzyme is provided. The method may include mixing an amount of animal feed with a carbonate-bicarbonate buffer to obtain a mixture. The animal feed may include a feed enzyme. The carbonate-bicarbonate buffer may include sodium carbonate and sodium bicarbonate. The concentration of sodium carbonate in the carbonate-bicarbonate buffer may be any concentration described herein. The concentration of sodium carbonate may be 30 mM. The concentration of sodium bicarbonate may be in the carbonate-bicarbonate buffer may be any concentration described herein. The concentration may be 30 mM. The method may include extracting the feed enzyme from the mixture.

In an embodiment, the feed enzyme may be any enzyme included in animal feed. The feed enzyme may be, but is not limited to, phytase, xylanase, glucanase, endoglucanase, cellobiohydrolase, amylase, protease, mannanase, arabinofuranosidase, xylosidase, glucoamylase, pectinase, lignin peroxidase, esterase, or cellulase. The phytase may be any phytase described herein. The phytase may be E. coli phytase.

In an embodiment, the feed enzyme may be produced in a genetically engineered host. The host may be, but is not limited to, a plant cell, a bacterial cell, a mammalian cell, and a yeast cell.

A temperature of the mixture during the step of extracting may be any temperature described in methods herein. The temperature may be 55° C. A pH of the carbonate-bicarbonate buffer may be any pH described in the methods herein. The pH may be 10.00, or greater.

The amount of animal feed may be in any amount used in the methods described herein. The amount of the animal feed may be at least 100 g. In an embodiment, the method may further comprise grinding the animal feed to form flour prior to the step of mixing. The flour may comprise particles having any size described in the methods herein. The size of the particles may be at least 250 μm. The amount of the animal feed may be the amount of flour produced after grinding. The amount of flour may be any amount of flour used in the methods described herein. The amount of flour may be at least 1 g. The carbonate-bicarbonate buffer may further comprise a nonionic detergent. The nonionic detergent may be any nonionic detergent described herein. The nonionic detergent may be TWEEN 20®. The concentration of TWEEN 20® in the carbonate-bicarbonate buffer may be in a range from 0.001% (v/v) to 1.0% (v/v), endpoints inclusive, or any concentration of TWEEN 20® in the carbonate-bicarbonate buffer described herein.

In an embodiment, the mixture may comprise the amount of feed and the carbonate-bicarbonate buffer at a ratio of less or equal to one selected from the group consisting of: 1:5 (w/v), 1:10 (w/v), 1:20 (w/v), 1:50 (w/v), 1:60 (w/v), 1:70 (w/v), 1:80 (w/v), 1:90 (w/v), 1:100 (w/v), 1:200 (w/v), 1:300 (w/v), 1:400 (w/v), 1:500 (w/v), 1:600 (w/v), 1:700 (w/v), 1:800 (w/v), 1:900 (w/v) and 1:1000 (w/v), or any value between any two of the foregoing value points.

The following list includes particular embodiments of the present invention. But the list is not limiting and does not exclude alternate embodiments, as would be appreciated by one of ordinary skill in the art.

### EMBODIMENTS

1. A method for measuring activity of phytase in an animal feed comprising:

mixing an amount of an animal feed with a carbonate-bicarbonate buffer to obtain a mixture, wherein the animal feed includes phytase, and the carbonate-bicarbonate buffer includes sodium carbonate at a concentration in a range from 10 mM to 500 mM and sodium bicarbonate at a concentration in a range from 10 mM to 500 mM;

extracting the phytase from the mixture; and

measuring the activity of an extracted phytase.

2. The method of embodiment 1 further comprising grinding an animal feed to form flour prior to the step of mixing.
3. The method of embodiment 2, wherein the flour comprises particles having a size within a range of 250 μm to 6,000 μm.
4. The method of embodiment 3, wherein the size is at least 250 μm.
5. The method of any one or more of the preceding embodiments, wherein a pH of the carbonate-bicarbonate buffer is 10.00, or greater.
6. The method of any one or more of the preceding embodiments, wherein the carbonate-bicarbonate buffer further comprises a nonionic detergent.
7. The method of any one or more of the preceding embodiments, wherein the amount of animal feed is within a range from 100 g to 500 g.
8. The method of any one or more of the preceding embodiments, wherein a temperature of the mixture is in a range 20° C.-80° C.
9. The method of any one or more of the preceding embodiments, wherein phytase is produced in a genetically engineered host.
10. The method of embodiment 9, wherein the host is selected from a plant cell, a bacterial cell, a mammalian cell, and a yeast cell.
11. The method of any one or more of the preceding embodiments, wherein the phytase is an E. coli phytase.
12. The method of any one or more of the preceding embodiments, wherein measuring of the phytase activity is performed at a temperature within a range of 20° C.-80° C.
13. The method of any one or more of the preceding embodiments, wherein the temperature is 37° C.
14. A method for extracting a feed enzyme comprising:

mixing an amount of animal feed with a carbonate-bicarbonate buffer to obtain a mixture, wherein the animal feed includes a feed enzyme, and the carbonate-bicarbonate buffer includes sodium carbonate at a concentration in a range from 10 mM to 500 mM and sodium bicarbonate at a concentration in a range from 10 mM to 500 mM; and

extracting the feed enzyme from the mixture.

15. The method of embodiment 14, wherein a pH of the carbonate-bicarbonate buffer is 10.00, or greater.
16. The method of any one or both of embodiments 14-15, wherein the carbonate-bicarbonate buffer further comprises a nonionic detergent.
17. The method of embodiment 16, wherein the nonionic detergent is a polysorbate.
18. The method of embodiment 17, wherein the polysorbate is at a concentration in a range from 0.001% (v/v) to 1.0% (v/v).
19. The method of any one or both of embodiments 14-18, wherein the mixture comprises the amount of feed and the carbonate-bicarbonate buffer at a ratio of less or equal to one selected from the group consisting of: 1:5 (w/v), 1:10 (w/v), 1:20 (w/v), 1:50 (w/v), 1:60 (w/v), 1:70 (w/v), 1:80 (w/v), 1:90 (w/v), 1:100 (w/v), 1:200 (w/v), 1:300 (w/v), 1:400 (w/v), 1:500 (w/v), 1:600 (w/v), 1:700 (w/v), 1:800 (w/v), 1:900 (w/v) and 1:1000 (w/v).
20. The method of any one or both of embodiments 14-19, wherein the amount of animal feed is within a range from 100 g to 500 g.
21. The method of any one or both of embodiments 14-20, further comprising grinding the animal feed to form flour, wherein the flour comprises particles having a size within a range of 250 μm to 6,000 μm.
22. The method of embodiment 21, wherein the size is at least 250 μm.
23. The method of any one or both of embodiments 14-22, wherein a temperature of the mixture is in a range 20° C.-80° C.
24. The method of embodiment 23, wherein the temperature is 50° C.
25. The method of any one or both of embodiments 14-24, further comprising measuring the activity of the feed enzyme.
26. The method of any one or both of embodiments 14-25, wherein the feed enzyme is produced in a genetically engineered host.
27. The method of embodiment 26, wherein the host is selected from a plant cell, a bacterial cell, a mammalian cell, and a yeast cell.
28. The method of any one or both of embodiments 14-27, wherein the feed enzyme is selected from the group comprising phytase, xylanase, glucanase, endoglucanase, cellobiohydrolase, amylase, protease, mannanase, arabinofuranosidase, xylosidase, glucoamylase, pectinase, lignin peroxidase, esterase, or cellulase.
29. The method of embodiment 28, wherein the phytase is an E. coli phytase.
30. The method of embodiment 25, wherein the step measuring is performed at a temperature within a range of 30° C.-80° C.
31. The method of embodiment 30, wherein the temperature is 37° C.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1. Phytase Extraction and Activity Assay

The method includes a procedure for phytase extraction from feed samples for use in the activity assay. The extraction procedure uses 30 mM sodium carbonate-bicarbonate buffer, pH 10.8, to extract the phytase protein at a temperature ranging from 23° C. to 75° C., and maximize extraction of phytase from feed with minimum background (caused by high phosphate in feed, endogenous phosphate and/or other enzymes). The assay is especially useful for the detection of phytase in feed supplemented with high levels of phosphate and the detection in feed of non-phytase proteins. It was observed that increasing extraction volume and adjusting the ratio of feed weight to extraction volume, such as 1/15 (w/v) to 1/30 (W/v), increased the efficiency of phytase extraction. It was also noticed that extending the extraction time, or repetitive extractions, such as an initial 1 hour extraction followed by an additional 1 hour extraction and mixing with fresh extraction buffer can maximize extraction of phytase. Addition of TWEEN 20® to the sodium carbonate-bicarbonate buffer further enhances the phytase extraction efficiency.

The phytase activity assay detects the presence of inorganic phosphate released from the sodium phytate substrate. The activity assay performed at an elevated temperature, i.e., 65° C., maximizes the assay sensitivity and allows accurate measurement of the phytase activity in feed, where the enzyme is typically diluted by a few thousand-fold.

Equipment and Consumables

TABLE 1
Equipment and consumables

| | |
|---|---|
| Udy mill | Udy Coporation, Model 3010-080P |
| 0.5 mm screen | UDY Corporation, Cat.: No. 30-0471 |
| 1.0 mm screen | UDY Corporation, Cat.: No. 30-0472 |
| Cutting mill | Retsch SM100 |
| 6.0 mm screen | GlenMills, Cat. No. : 1616-002516 |
| 1.0 mm screen | GlenMills, Cat. No. : 1616-002512 |
| Roller mill | Roskamp Champion, Model 113650-9 |
| Glass Collection Bottles | UDY Corporation, Cat. No. : 35-0389/90 |
| with snap cap 120 ml | |
| Vacuum | Stinger, Model WD20250 |
| Brush for Udy Mill | Karter Scientific 210W2 Test Tube |
| | Bottle Brushes |
| Polypropylene beaker | Fisher Scientific, Cat.: No. 02-591-32 |
| Bucket | Home Depot, Model 05GLHD2 |
| Analytic balance | Mettler, Model PM 4000 |
| Pipetman (10-300 μL | Gilson pipetman |
| multichannel, 100- | |
| 1200 μl multichannel, | |
| 20 μl, 100 μl and 200 μl) | |
| Shaker | New Brunswick, Model INNOVA 4300 |
| Incubator set at 55° C. | Hybaid, Model H9360 |
| with rotation platform | |
| Incubator set at 65° C. | Hybaid, Model H9360 |
| Incubator set at 37° C. | Heraeus, Model D-63450 |
| Centrifuge for spinning | Beckman Coulter, Model Alegra 25R, |
| down 96 well plates | Rotor S5700 |
| Benchtop centrifuge | Beckman Coulter, Model Alegra 6R, |
| | Rotor GH-3.8 |
| Vortex mixer | Scientific Industries, Model G-560 |
| pH meter | Thermo Scientific, Model Orion 3 Star |
| Micro plate reader | Bio-Tek, Model Synergy HT |
| capable of reading | |
| 415 nm | |
| Hot plate/Stirrer | VWR, Cat. No. : 97042-602 |
| 1L glass bottles* | Corning, Cat. No. : 13951L |
| 500 mL glass bottles* | Corning, Cat. No. : 13951-500 |
| Pipette tips 1-200 μL | Fisher, Cat. No. : 02-707-420 |
| and 100-1200μL | Rainin, Cat No.: RT-L1200 |
| Serological pipettes 5 | Falcon Cat Nos: 356543 and 357525 |
| and 25 ml | |
| 50 mL Reagent | Corning-Costar, Cat. No. : 4870 |
| Reservoirs | |
| Adhesive sealing film | Sigma, Cat. No. : Z369675 |
| 2 mL 96 well assay | Costar, Cat. No. : 3960 |
| block | |
| Round bottom 96 well | Costar, Cat. No. : 3797 |
| plates | |
| Flat bottom 96 well | Costar, Cat. No. :9017 |
| plates | |
| Aluminum foil | Fisher Scientific, No. 01-213-103 |
| Plastic bags | Reloc Zippit ® 4″ x 6″ 2 Mil Resealable |
| | Bags with White Block |
| 50 mL Falcon conical | BD Falcon, Cat. No. : 352070 |
| centrifuge tubes | |
| 2 mL Falcon conical | BD Falcon, Cat. No. : 352096 |
| centrifuge tubes | |
| Flask (250 mL, 500 mL, | Nalgene, Cat. No. : 4112-0250, 4112- |
| 1000 mL, 2800 mL) | 500, 4112-1000, 4112-2800 |
| Lab Timer | VWR, Cat. No. : 62344-641 |
| Plastic spoon | Home Depot, Mode FG288400CLR |
| Face marks | Defend MK 1046 |

* All glassware was rinsed with nitric acid, followed by extensive washes with dH₂0 to remove any residual phosphate.

TABLE 2
Reagents and chemicals

| | |
|---|---|
| TWEEN 20 ® | Fisher, Cat. No. : BP337-100 |
| Sodium acetate, | Sigma, Cat. No. : S8750-1 kg |
| anhydrous | |
| Calcium chloride, | Acros Organics, Cat. No. : |
| dehydrate | 423525000 |
| Acetic acid, glacial | Fisher, Cat. No. : BP2401SI-212, |
| | or equivalent |
| Phytic Acid | Biosynth International, |
| dodecasoclium salt, | Cat. No. : P-6700 |
| 80% | |
| Ammonium molybdate | Fisher, Cat. No. : A674-500 |
| tetrahydrate | |
| Ammonium hydroxide | Fisher, Cat. No. : A6695-500 |
| solution, 29% w/v | |
| Ammonium | Sigma, Cat. No. : 398128-50G |
| metavanadate | |
| Nitric acid, | Fisher, Cat. No. : A200-500 |
| concentrated >65% | |
| Potassium phosphate | Fisher, Cat. No. : P286-1 |
| monobasic | |
| Sodium carbonate | Sigma, Cat. No. : 223530-500G |
| Sodium bicarbonate | Fisher, Cat. No. : S233-500 |
| Sodium tetraborate | Sigma, Cat. No. : B9876-500G |
| decahydrate | |

Experimental Preparations

All buffers and solutions were made using labware utilized only for phytase preparation.

Diluted Nitric Acid:

was prepared by adding 70 mL of concentrated nitric acid to 130 mL of dH₂O in a glass bottle.

10% TWEEN 20®:

was prepared by dissolving 5 mL of TWEEN 20® in 45 mL of dH₂O.

Ammonium Molybdate Stock Solution:

was prepared by dissolving 50 g of ammonium molybdate tetrahydrate in 400 mL of dH₂O, adding 5 mL of ammonium hydroxide, and filling into a 500 mL glass bottle. The bottle was wrapped with aluminum foil to shield from light and was stored at room temperature in a dark place for up to 90 days.

Ammonium Vanadate Stock Solution for Color Stop Solution:

was prepared by dissolving 1.175 g of ammonium vanadate in 400 mL of dH₂O, and heating the sample in a 60° C. water bath to aid dissolution. The solution starts turning yellow as the compound dissolves. Once the ammonium vanadate is completely dissolved, 10 mL of diluted nitric acid was slowly added to the solution while stirring. The solution was cooled to room temperature, transferred to a glass bottle, and adjusted to the mark 500 mL with dH₂O. The bottle was wrapped with aluminum foil to shield from light and was stored at room temperature in a dark place for up to 90 days.

Sodium Acetate Buffer, 250 mM, pH 5.5 with 1 mM Calcium Chloride and 0.01% TWEEN 20®:

was prepared by dissolving 18.096 g of sodium acetate in 600 mL of deionized water. The pH of the sample was adjusted to 5.5 using 1.676 mL acetic acid. 0.147 g calcium chloride was dissolved in this solution and mixed with 1.0 mL of 10% TWEEN 20®, maintaining pH at 5.5. The solution was adjusted to the 1000 mL mark with dH₂O, and stored at 4° C. for up to 90 days.

Potassium Phosphate Standard, 7.2 mM, pH 5.5:

was prepared by weighing out 0.049 g potassium phosphate monobasic and dissolving in 50 mL sodium acetate buffer in a 50 mL Falcon tube. The pH was confirmed to be 5.5. The standard was stored at 4° C. for up to 90 days

Sodium Phytate Substrate, 9.1 mM, pH 5.5:

was prepared fresh daily by dissolving 0.2102 g of phytic acid into 25 mL sodium acetate buffer, pH 5.5, which was sufficient for a full 96-well plate assay plus phosphate standard curve preparation.

Sodium Carbonate/Bicarbonate Extraction Buffer, 30 mM, pH10.8

(1) 100 mM sodium carbonate was prepared by weighing out 5.30 g of sodium carbonate, dissolving it in distilled water and adjusting the final volume to 500 mL.

(2) 100 mM sodium bicarbonate was prepared by weighing out 4.2 g of sodium bicarbonate, dissolving it into distilled water and adjusting the final volume to 500 mL.

(3) 450 ml of 100 mM sodium carbonate was premixed with 50 mL of 100 mM sodium bicarbonate to bring pH to 10.8 at 100 mM. 300 mL of premix was diluted with water to final 1000 mL. pH was adjusted to 10.8 with 1M NaOH if needed.

Sodium Carbonate/Bicarbonate Extraction Buffer, 30 mM, pH10.8, 0.01% TWEEN 20®.

One thousand milliliters of the sodium carbonate/biocarbonate buffer was prepared as stated above. One milliliter of 10% TWEEN 20® was added to 1000 mL sodium carbonate/bicarbonate buffer. The sodium carbonate/biocarbonate buffer was stored at 4° C. for up to 90 days.

Sodium Borate Extraction Buffer, 25 mM, ph 10.0, 0.01% TWEEN 20®. 9.534 g of sodium borate was dissolved in 600 mL of deionized water in a glass bottle, and 1.0 mL of 10% TWEEN 20® solution was added. The pH was adjusted to 10 using 3.8 mL of 10 N sodium hydroxide. The volume was adjusted to 1000 mL with deionized water.

Color Stop Solution:

was prepared during 1 hour enzyme incubation time at 37° C. and was kept in the dark before using it. The components listed below were added together in a 50 mL Falcon tube for a total 25 mL of the Color Stop Solution.

TABLE 3
Components of the Color Stop Solution

| Component | Volume (mL) |
|---|---|
| dH₂O | 8.375 |
| Ammonium Molybdate | 6.25 |
| Stock Solution | |
| Ammonium Vanadate | 6.25 |
| Stock Solution | |
| 35% Nitric Acid | 4.125 |

The color of the stop solution was faint yellow.

### Example 2. Experimental Procedures

(1) Mill Grain or Feed Samples:

Mixing Feed Samples.

Feed was pooled in a 5 gallon bucket. Feed was stirred with a plastic spoon to mix feed well before milling. Feed samples of 100 g up to 500 g are taken from the pooled feed in the 5 gallon bucket. The most commonly used feed sample size was 250 g.

Feed samples were milled by using Udy mill with 0.5 mm screen, or Retsch SM100 cutting mill with 1.0 mm screen, or Roskamp Champion TP650-9 roller mill and sieved to desired particle sizes. Flour was stored in a labeled bag and kept in a dry cool area.

The mill was cleaned between each sample using brush and vacuum, then blown with air.

(2) Protein Extraction from Feed Samples:

5 g, 10 g, 20 g or 100 g of milled feed sample were weighed out in 250 ml, 500 ml, 1000 ml or 2800 ml flasks.

Extraction buffer was added to the flour and vortexed aggressively to suspend the flour. In the assay, 20 g flour was used for phytase protein extraction. The flasks were loaded to a shaker and were shaking under 250 rpm at 23° C. or 55° C. for 1 hour. After 1 hour, samples were removed from the shaker, 1.5 ml suspension were taken to a 2 ml tube and spinned down at 16000× g for 10 minutes in a benchtop centrifuge. The supernatant including the protein was collected into a 2 ml tube and used for further analysis. If feed samples required further extraction for the maximal recovery, a fresh extraction buffer was added to the feed suspension based on the w/v ratio. For example, if the w/v ratio was 5, 100 ml extract buffer had to be added to the flask; if the w/v ratio was 10, then 200 ml buffer had to be added to the flask. After mixing with fresh extraction buffer, the flasks were placed at the shaker for additional time at the selected temperatures.

Dilution of the Protein Extract from Feed Samples:

the phytase protein extract from feed was diluted with the 250 mM sodium acetate buffer (pH 5.5, 1 mM calcium chloride and 0.01% TWEEN 20®). The typical dilution was from 5-fold and up to 40-fold depending on the phytase inclusion in the feed. The tested dilutions of the protein extract from feed were 5, 10, 20 and 40. It was observed that the phytase protein extract has to be sufficiently diluted for the inorganic phosphate released by phytase to be detected, that is, to be within the linear detection range of the phosphate standard curve.

Phytase Activity Assay

9.1 mM sodium phytate, the substrate for the phytase activity assay, was prepared in advance as described herein. A 96-deep-well assay block used for the test included 12 columns and 8 rows labeled A, B, C, D, E, F, G, and H. Seventy five microliters of the diluted phytase extract were transferred to the wells in rows A to D (Reaction) of the assay block and the sample information of each well was recorded. 5-fold, 10-fold, 20-fold and 40-fold dilutions were tested. The sodium phytate substrate was pooled to the sample tray and covered with an aluminum foil. The assay block containing samples was sealed. Both phytate substrate and the assay block were incubated at 37° C. or 65° C. for 10 minutes. One hundred fifty microliters of the pre-warmed 9.1 mM phytic acid substrate were first added to rows E-H (Blank), where the phytase protein extract was not included. Then, one hundred fifty microliters of the pre-warmed 9.1 mM phytic acid substrate were added to the row A and well mixed. Finally, one hundred fifty microliters of the pre-warmed 9.1 mM phytic acid substrate were added to the remaining rows B, C, and D. The 96 assay block was sealed and incubated at 37° C. or 65° C. per for 60 minutes.

The Color Stop solution was prepared as described in Example 1 herein. After 60 minutes incubation, 150 μL of the Color Stop solution were added to the 96 assay block in a fume hood, starting at row A and well mixed, then 150 μL of the Color Stop solution were added to the remaining rows of B, C, D, E, F, G, and H to stop the reaction. For the blank rows E-H, 75 μL of the corresponding dilutions of the phytase sample extract were added after the Color Stop Solution, so that no phosphorus was produced by phytase. Since the blank rows contained the same amount of substrate, the phytase sample extract and the Color Stop Solution, blank rows can be used to obtain the background phosphate reading. The assay block was placed in the fume hood for 10 minutes, and centrifuged at 3000×g for 10 minutes. One hundred microliters of the supernatant from each well of the assay block were transferred to a flat bottom microplate. Optical density of the samples in the microplate was read at 415 nm.

Phosphate Standard Curve

A phosphate standard curve was prepared each time a set of assays was performed, so the inorganic phosphorus released from phytic acid substrate by the activity of phytase can be quantitated using the standard curve. Each phytase standard was made by mixing 250 mM sodium acetate buffer with 7.2 mM potassium phosphate prepared as described herein. The volumes of potassium phosphate and sodium acetate used to make each standard, such as listed in Table 4, were aliquoted into a round bottom 96 well plate from column 1 to column 12 as the phosphate standard of 1 to 12.

One hundred microliters of the phytic acid substrate were added to each phosphate standard, and mixed. Then 100 μl Color Stop Solution was added and mixed. The mixtures were incubated for 10 minutes at room temperature to complete the color reaction, followed by centrifugation at 3000× g for 10 minutes to clarify the solution.

One hundred microliters of the supernatant from each standard mix was transferred to a new flat bottom microplate for absorbance measurement at 415 nm (OD₄₁₅).

TABLE 4
Aliquots of the phosphate/buffer volumes

| Standard (Std) | 1 | 2 | 3 | 4 | 5 | 6 | 7 | 8 | 9 | 10 | 11 | 12 |
|---|---|---|---|---|---|---|---|---|---|---|---|---|
| Sodium Acetate | 50 | 45 | 42.5 | 40 | 37.5 | 35 | 25 | 20 | 15 | 10 | 5 | 0 |
| Buffer (μL) | | | | | | | | | | | | |
| 7.2 mM Potassium | 0 | 5 | 7.5 | 10 | 12.5 | 15 | 25 | 30 | 35 | 40 | 45 | 50 |
| Phosphate (μL) | | | | | | | | | | | | |
| Potassium phosphate | 0 | 144 | 216 | 288 | 360 | 432 | 720 | 864 | 1008 | 1152 | 1296 | 1440 |
| concentration (μM) | | | | | | | | | | | | |

Expression of Phytase Activity

Phytase activity was determined as described in AOAC Official Method 2000.12, Phytase Activity in Feed, Colorimetric Enzymatic Method, First Action 2000; and U.S. Pat. No. 7,629,139, issued Dec. 8, 2009, all of which are incorporated herein by reference as if fully set forth.

Phosphate Standard Curve:

the OD₄₁₅ reading of standard 1 (0 μM phosphate) was subtracted from each 415 nm measurement (ΔOD₄₁₅) for correcting the phosphate standard curve readings by subtracting a reagent blank.

The corrected absorbance at 415 nm (ΔOD₄₁₅) was plotted on Y-axis as a function of phosphate concentration (in μM) on the X-axis, and then the “best fit” line and its corresponding equation was calculated through the data set using linear regression:

X=(Y−R)/Z,

where X=μM of phosphate standard (X-axis of the plot)

Y=ΔOD₄₁₅ (Y-axis of the plot)

R=intercept of the phosphate standard calibration curve

Z=slope of the phosphate calibration standard curve

Phytase Activity Calculation

Phytase activity was calculated in the protein extract sample based on the concentration of inorganic phosphorus released from the phytase reaction.

One phytase unit (FTU) is defined as that quantity of enzyme that will liberate 1 μmol of inorganic phosphorus per minute under the conditions of the assay. In the assay, the phytase enzyme was incubated with the substrate phytic acid in sodium acetate buffer pH 5.5, at 37° C. or 65° C. for 60 minutes.

Blank measurements of the protein extract samples (row E to H) were subtracted from their corresponding reaction measurements (row A to D), for which the same diluted phytase protein extract (ΔOD₄₁₅′) was used. The corrected background absorbance (ΔOD₄₁₅′, such as Y in the previous formula) was used to determine phosphate values using the phosphate standard curve regression parameters. The value was calculated in μM (X′, such as X in the previous formula).

Concentration of organic phosphate released by phytase in samples was determined using the following equation:

X′=(Q−R)/Z,

where, X′=concentration (μM) of phosphate produced by phytase activity

Q=ΔOD₄₁₅′

R=intercept of the phosphate standard calibration curve

Z=slope of the phosphate calibration standard curve

Phytase activity in samples was calculated using the following equation:

P=[(X′×Vm/Vs)×(Ve/1000)×Di]/(Ti×DW),

where,
P=phytase activity (FTU/g) expressed in μmols of inorganic phosphorus released per minute per gram of feed sample
X′=concentration (μM) of organic phosphate produced by phytase activity; as described as above, X′=(Q−R)/Z
Vm=total volume (in mL) of the reaction mixture after addition of the color stop solution. Per present study, it is 0.375 mL
Vs=Volume (in mL) of the diluted sample extract including phytase used for each assay. Per present study, it is 0.075 mL
Ve=Buffer volume (in mL) used to extract protein. In this assay, Ve was 50, 100, 150, 200, 250, or 300 mL.
(Ve/1000)=buffer volume (in liter) used to extract protein. In this assay, the buffer volume was 0.05, 0.1, 0.15, 0.2, 0.25, or 0.3 L
Di=working solution dilution factor [Di=Vf/Vp, where, Vf=final volume (or mass, assuming density to be equal to 1) of total dilution mix;
Vp=volume (or mass, assuming density to be equal to 1) of extracted protein used for working dilution. In this assay, Di was 5, 10, 20, or 40 for feed sample assay.
Ti=incubation period (in minutes) during the phytase assay. In this assay, Ti was 60 minutes.
DW=initial mass (in grams) of sample used for each extraction. In this assay, DW was 5, 10, 20, or 100 g.

### Example 3. Buffer Developed for Efficient Extraction of Phytase from Feed and Accuracy of Activity Assay

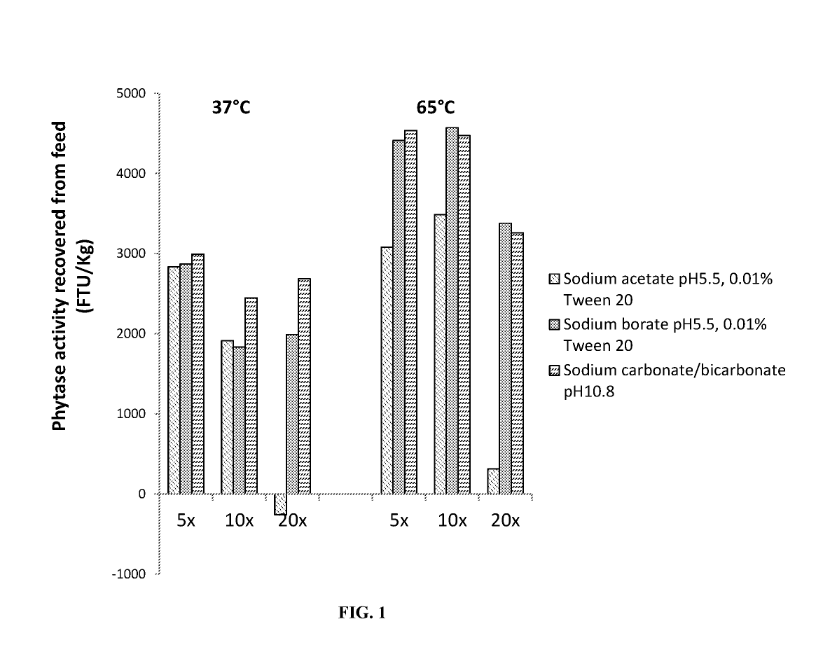

FIG. 1 illustrates comparison of the efficiency of phytase extraction by using the sodium acetate buffer, pH 5.5, 0.01% TWEEN 20®; the sodium borate buffer, pH 10, 0.01% TWEEN 20®; and the sodium carbonate-bicarbonate buffer, pH 10.8. Referring to FIG. 1, the extraction procedure was performed at 23° C. using all three buffers and the phytase activity was assayed at 37° C. or 65° C. It was observed that phytase activity was most efficiently recovered from mash feed using the sodium carbonate-bicarbonate buffer when the assay was performed at 37° C. When an assay was performed at 65° C., the sodium borate buffer and the sodium carbonate/bicarbonate buffer were similarly efficient in recovery of phytase activity, and more efficient than the sodium acetate buffer, pH 5.5.

When phytase activity was assayed at 65° C., the sodium borate buffer and the sodium carbonate/bicarbonate buffer showed similar efficacy in recovery of phytase activity, and were better than the sodium acetate buffer, pH 5.5. The effect of extract dilution on recovery of enzymatic activity was also tested for three buffers. Referring to FIG. 1, the protein extract was diluted with 250 mM sodium acetate buffer (pH 5.5, 0.01% TWEEN 20®, 1 mM sodium chloride) before incubation with phytic acid at 37° C. or 65° C. for 1 h.

Phytase was diluted and assayed at 37° C. No significant difference in phytase activity was observed among three extraction buffers after 5-fold (low) dilution since inorganic phosphorus released by the concentrated phytase reached the plateau of the phosphate standard curve, and the detection of phosphate level became less sensitive. When the protein extract was further diluted to 10- or 20-fold (high), less phytase was added to the reaction, and phosphate produced by the enzyme was more easily detectable, and the assay became more sensitive. More phosphorus was detected in the reaction with the diluted protein from carbonate-bicarbonate extraction than with diluted protein from other buffers tested. It was observed that after high levels of dilution more phytase was extracted from feed at 37° C. by the carbonate-bicarbonate buffer than by the sodium borate buffer and the sodium acetate buffer.

Referring to FIG. 1, it was also observed that sodium borate buffer supplemented with 0.01% TWEEN 20® and sodium carbonate-bicarbonate buffer were similarly efficient in recovery of the phytase activity at 65° C. Recovery of the phytase activity with these buffers was more efficient than that with sodium acetate buffer, pH 5.5.

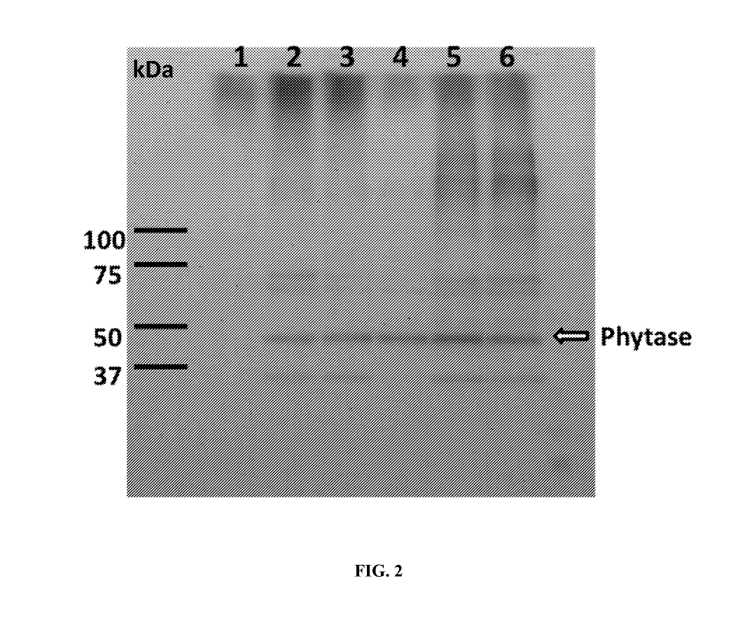

Data confirming that 30 mM sodium carbonate/bicarbonate buffer, pH 10.8, was efficient for extraction phytase from feed is illustrated in FIG. 2. FIG. 2 is a photograph of a Western blot showing that more phytase was extracted from feed using sodium carbonate/bicarbonate buffer, pH 10.8, than using sodium acetate buffer, pH 5.5. Less non-phytase protein bands were observed in the sodium carbonate/bicarbonate extract. It was observed that increasing temperature during protein extraction results in extraction of more phytase from feed. Referring to FIG. 2, lanes 1 and 4 correspond to protein extracted in 250 mM sodium acetate buffer, pH 5.5, 0.01% TWEEN 20® at 23° C. (lane 1) or at 55° C. (lane 4), lanes 2 and 5 correspond to protein extracted in 25 mM sodium borate buffer, pH 10, 0.01% TWEEN 20® at 23° C. (lane 2) or at 55° C. (lane 5), lanes 3 and 6 correspond to protein extracted in 30 mM sodium carbonate/bicarbonate buffer, pH 10.8, at 23° C. (lane 3) or at 55° C. (lane 6). Although the phytase extraction appeared similarly effective at 23° C. using carbonate/bicarbonate buffer and borate buffer, the carbonate/bicarbonate extract had lower background of non-specific proteins than the borate extract. Whether supplemented with phytase or not, feeds for monogastric animals are fortified with inorganic phosphate typically in the form of monocalcium phosphate or dicalcium phosphate. This inorganic phosphate component in the feed, in addition to phosphate present in the vegetable components of the feed, is not distinguishable from phosphate released by phytase activity during an enzymatic assay. Therefore, it is advantageous to minimize the background phosphate during extraction from feed while maximizing the phytase extracted protein. Achieving a low concentration of phosphate relative to the phytase concentration in a feed extract results in a higher signal-to-noise ratio for the phytase activity assay. Referring to FIGS. 1 and 2, the sodium carbonate/bicarbonate buffer, pH 10.8, recovers more phytase activity from feed compared to sodium acetate buffer, pH 5.5. The phytase activity recovered in carbonate/bicarbonate buffer, pH 10.8, was slightly higher than the activity recovered in borate buffer, pH 10, supplemented with 0.01% TWEEN 20®.

### Example 4. Increasing Protein Extraction Temperature Improves Efficiency of Phytase Extraction

Increasing protein extraction temperature helps extracting more phytase from feed for all three buffers tested.

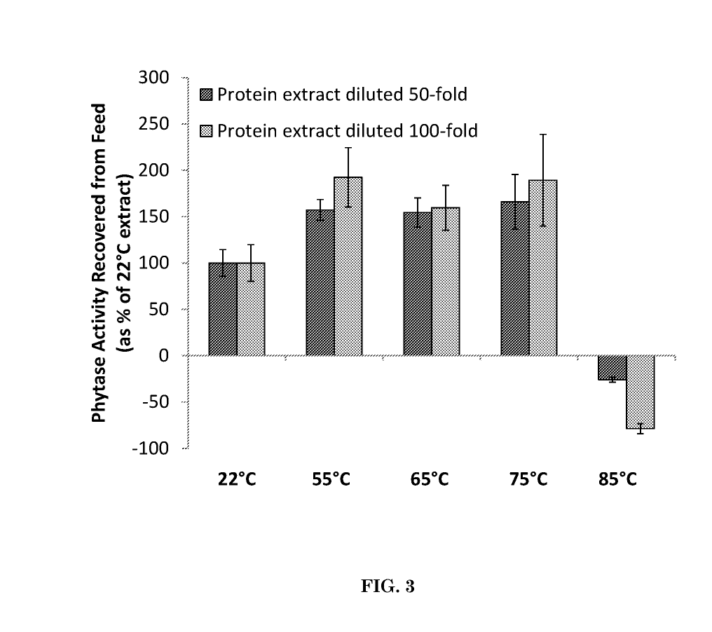

FIG. 3 illustrates efficiency in phytase extraction from feed samples using the sodium carbonate/bicarbonate buffer at 55° C., 65° C., 75° C., and 85° C. and 50-fold and 100-fold dilutions. FIG. 3 shows improvement in efficiency of phytase recovery at high temperatures 55° C., 65° C., and 75° C., compared to room temperature 22° C. The assay to determine phytase activity was performed at 37° C. for all treatments. In the assay, the protein extract was diluted in sodium acetate buffer, pH 5.5 before incubation with phytic acid at 37° C. for 1 hour.

Referring to FIG. 3, it was observed that the carbonate/bicarbonate buffer, pH 10.8, recovers more phytase activity when the protein extraction was carried out for one hour at high temperatures 55° C., 65° C. and 75° C. compared to room temperature 22° C. FIG. 3 also shows that phytase lost its activity after the protein was extracted at 85° C. for one hour.

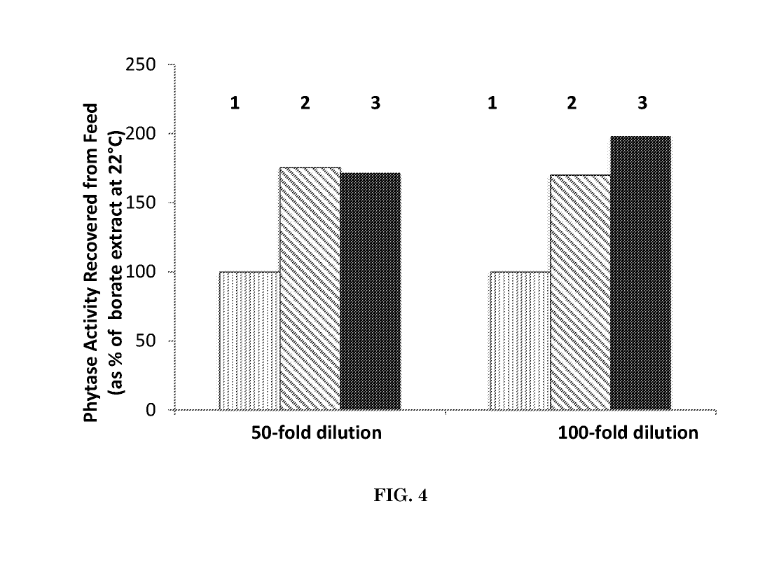

FIG. 4 illustrates phytase activity recovered from feed after extraction using the sodium borate buffer, pH 10, 0.01% TWEEN 20® (bars 1 and 2) and the sodium carbonate/bicarbonate buffer, pH 10.8 (bar 3). Referring to FIG. 4, left panels illustrate phytase activity recovered following 50-fold dilution of the protein extract; and right panels illustrate phytase activity recovered following 100-fold dilution of the protein extract. In FIG. 4, phytase activity recovered from feed is expressed as a percent of the activity recovered from the corresponding feed sample using the borate extraction buffer, pH 10 plus 0.01% TWEEN 20® at room temperature and assayed at 37° C. The protein extract was diluted with 250 mM sodium acetate, pH 5.5, 0.01% TWEEN 20®, 1 mM sodium chloride before incubation with phytic acid at 37° C. or 65° C. for 1 hour. Bar 1 illustrates results for 25 mM sodium borate, pH 10, 0.01% TWEEN 20®, protein extraction for 1 hour at 23° C., and activity assay performed for 1 hour at 37° C. Bar 2 illustrates results for 25 mM sodium borate sodium borate (Na₃BO₃), pH 10, 0.01% TWEEN 20®, protein extraction for 1 hour at 55° C., and activity assay performed for 1 hour at 65° C. Bar 3 illustrates results for 30 mM sodium carbonate/bicarbonate (Na₂CO₃/NaHCO₃), pH 10.8, protein extraction for 1 hour at 55° C., and activity assay performed for 1 hour at 65° C. It was observed that phytase recovery from feed was increased when the protein was extracted at 55° C. and assayed at 65° C. in comparison to the assay performed at 37° C. For 50-fold dilution, phytase activity after extraction with the carbonate/bicarbonate buffer was almost as high as that with the borate buffer (bar 2). When the feed samples contain high levels of phytase, the protein extracts have to be properly diluted for the phytase produced phosphates to be detected, and thus, for the efficient measurement of phytase activity. It was observed that for 100-fold dilution, phytase activity was the highest after extraction with the carbonate/bicarbonate buffer, and that this buffer extracts phytase more efficiently than any other buffer tested in the treatment.

### Example 5. Adjusted Ratio of Feed Weight to Volume of Extraction Buffer (W/V) Increases Phytase Recovery from Feed

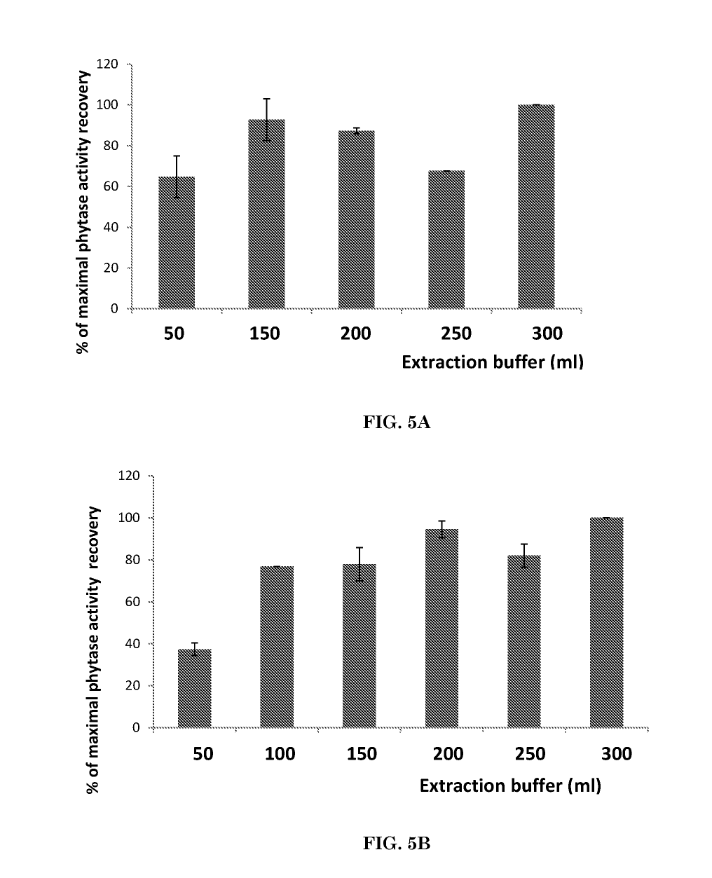

FIGS. 5A-5B illustrate percentage of the maximal activity recovered from 10 g feed formulated with 1000 FTU/kg (FIG. 5A) and 3000 FTU/kg (FIG. 5B) of phytase by using different volumes of sodium carbonate/bicarbonate extraction buffer. Phytase activity recovered from 300 mL extraction was defined as 100% (maximum phytase activity recovered). Referring to FIGS. 5A and 5B, 10 g feed formulated with 1000 FTU/kg (FIG. 5A) or with 3000 FTU/kg (FIG. 5B) was mixed in 50 mL (W/V=⅕), 100 mL (W/V= 1/10), 150 mL (W/V= 1/15), 200 mL (W/V= 1/20), 250 mL (W/V= 1/25), and 300 mL (W/V= 1/30) sodium carbonate/bicarbonate buffer, respectively, and was shaken under 250 rpm at 23° C. for 1 hour. Protein extract was diluted in sodium acetate pH 5.5 buffer, and tested at 37° C. for 1 hour. Referring to FIG. 5A, it was observed that upon inclusion in the feed of 1000 FTU/kg phytase, 50 mL buffer extracted 65% of the activity in the formulation. Referring to FIG. 5B, it was observed that upon inclusion in the feed of 3000 FTU/kg phytase, 50 mL buffer extracted 37% of the activity in the formulation. Referring to FIGS. 5A and 5B, it was observed that increasing volume of the extraction buffer to 150 mL resulted in extraction of 93% activity of the formulation dose 1000 FTU/kg and 78% activity of the formulation dose 3000 FTU/kg.

### Example 6. Particle Size of Flour/Feed Affects the Enzyme Recovery

Corn grains were milled and sieved to obtain three particle sizes: 1.0-1.4 mm, 1.7-2.36 mm, and 2.8-3.35 mm. Twenty grams of each ground sample were mixed with 100 mL AOAC Feed Buffer (220 mM sodium acetate, 69 mM calcium chloride, 0.01% TWEEN 20®), pH 5.5 (1) or carbonate/bicarbonate buffer (2) and were shaken at 250 rpm and 23° C. for 1 hour. Four milliliters of the solid/liquid mix were centrifuged and the supernatant was used for analysis of the phytase activity (initial extraction). The remaining solid/liquid mix was stored at 4° C. overnight, and then mixed with additional 96 mL carbonate/bicarbonate buffer. The solid/liquid mix was shaken at 250 rpm and 23° C. for another hour before precipitating the solid and using the supernatant for testing the phytase activity (second extraction).

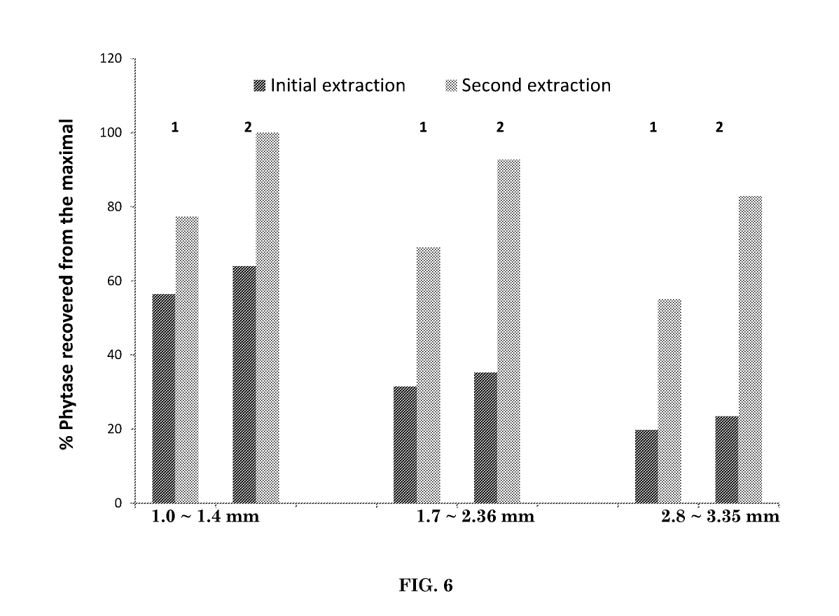

FIG. 6 illustrates the phytase activity recovered from transgenic flour ground into three different particle sizes at two extractions using (1) sodium acetate buffer and (2) sodium carbonate/bicarbonate buffer. Each of the two extractions were performed for 1 hour at 23° C. Prior to the second extraction, 96 mL of the extraction buffer were added to the initial extract.

Referring to FIG. 6, it was observed that phytase activity recovered from large size particles (2.8-3.35 mm) by two buffers at the initial extraction was lower compared to the maximal activity recovered at the second extraction. For example, for large size particles initial extraction with sodium carbonate/bicarbonate buffer (2) was only up to 24% of the maximal activity recovered at the second extraction. Phytase recovery from medium size particles (1.7-2.36 mm) was less than 35% of the maximal activity recovered at the second extraction. With additional extraction buffer and additional hour of extraction, buffer 1 and buffer 2 recovered, respectively, 55% and 83% of phytase activity from large size particles; and 69% and 93% of phytase activity from medium size particles. Sodium carbonate/bicarbonate buffer was observed to be more effective in phytase recovery from the enzyme containing particles of small sizes.

### Example 7. Including TWEEN 20® in Sodium Carbonate/Bicarbonate Buffer Increases Recovery of Phytase Activity from Feed

Addition of TWEEN 20® to sodium carbonate/bicarbonate buffer was tested for further increasing efficiency of phytase extraction.

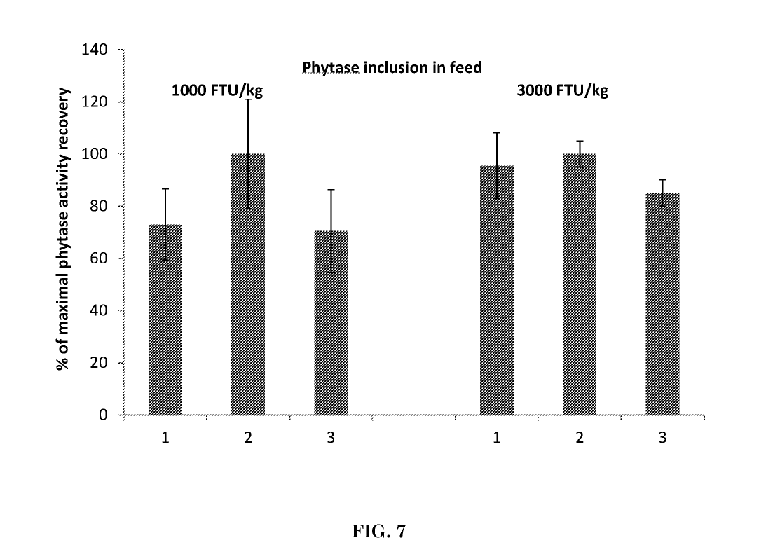

FIG. 7 illustrates the phytase activity recovered from the feed formulated with 1000 FTU/kg and 3000 FTU/kg phytase using sodium carbonate/bicarbonate buffer (1), sodium carbonate/bicarbonate buffer with 0.01% TWEEN 20® (2), and sodium borate buffer with 0.01% TWEEN 20® (3). Referring to FIG. 7, it was observed that the sodium carbonate/bicarbonate buffer (1) recovered more phytase from feed formulated with 3000 FTU/kg phytase than the sodium borate buffer supplemented with TWEEN 20® (3). The sodium carbonate/bicarbonate buffer (1) was equally efficient with the sodium borate buffer (3) in recovery of phytase activity from feed with low phytase inclusion, such as 1000 FTU/kg. Addition of TWEEN 20® to the sodium carbonate/bicarbonate buffer (2) improved efficiency of phytase extraction from feed with low phytase inclusion compared to the extraction with the sodium borate buffer (3).

### Example 8. Phytase Activity is Stable in Carbonate/Bicarbonate Buffer

Liquid enzyme application post pelleting is advantageous due to bringing uniformity of enzyme in feed, and preventing losses of enzyme activity during pelleting process that may exceed temperature of 85° C.

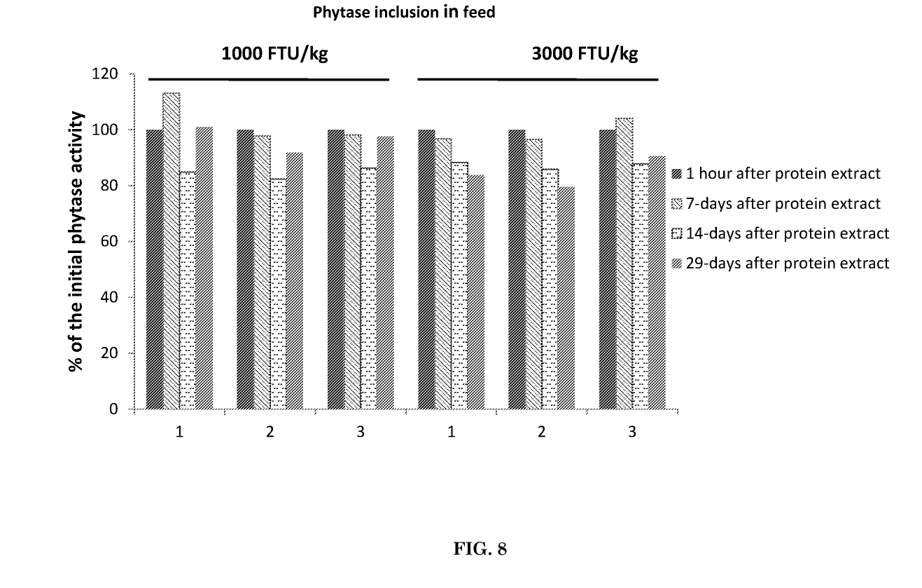

After extraction, the phytase extract was stored in the protein extraction buffers for extended period at 4° C., and then the enzyme activity was tested. FIG. 8 illustrates stability of the phytase activity following storage in the sodium carbonate/bicarbonate buffer with or without TWEEN 20®, or in the sodium borate buffer with TWEEN 20®, for 1 hour (black bar), 7 days (diagonally stripped bar), 14 days (horizontally partially stripped bar) and 29 days (gray bar). In FIG. 8, (1) refers to 30 mM sodium carbonate/bicarbonate, pH 10; (2) refers to 30 mM sodium carbonate/bicarbonate, pH 10.8, 0.01% Tween 20; and (3) refers to 25 mM sodium borate, pH 10.0, 0.01% Tween 20. Referring to FIG. 8, it was observed that no phytase activity was lost after 7-days storage in these buffers. It was also observed that approximately 12% to 23% activity was lost after 21-day storage in any of the buffers.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A method for measuring activity of phytase in an animal feed comprising:
mixing an amount of an animal feed with a carbonate-bicarbonate buffer to obtain a mixture, wherein the animal feed includes phytase, and the carbonate-bicarbonate buffer includes sodium carbonate at a concentration in a range from 10 mM to 500 mM and sodium bicarbonate at a concentration in a range from 10 mM to 500 mM;
extracting the phytase from the mixture; and
measuring the activity of an extracted phytase.

2. The method of claim 1 further comprising grinding an animal feed to form flour prior to the step of mixing.

3. The method of claim 2, wherein the flour comprises particles having a size within a range of 250 μm to 6,000 μm.

4. The method of claim 3, wherein the size is at least 250 μm.

5. The method of claim 1, wherein a pH of the carbonate-bicarbonate buffer is 10.00, or greater.

6. The method of claim 1, wherein the carbonate-bicarbonate buffer further comprises a nonionic detergent.

7. The method of claim 1, wherein the amount of animal feed is within a range from 100 g to 500 g.

8. The method of claim 1, wherein a temperature of the mixture is in a range 20° C.-80° C.

9. The method of claim 1, wherein phytase is produced in a genetically engineered host.

10. The method of claim 9, wherein the host is selected from a plant cell, a bacterial cell, a mammalian cell, and a yeast cell.

11. The method of claim 1, wherein the phytase is an E. coli phytase.

12. The method of claim 1, wherein measuring of the phytase activity is performed at a temperature within a range of 20° C.-80° C.

13. The method of claim 12, wherein the temperature is 37° C.

14. A method for extracting a feed enzyme comprising:
mixing an amount of animal feed with a carbonate-bicarbonate buffer to obtain a mixture, wherein the animal feed includes a feed enzyme, and the carbonate-bicarbonate buffer includes sodium carbonate at a concentration in a range from 10 mM to 500 mM and sodium bicarbonate at a concentration in a range from 10 mM to 500 mM; and
extracting the feed enzyme from the mixture.

15. The method of claim 14, wherein a pH of the carbonate-bicarbonate buffer is 10.00, or greater.

16. The method of claim 14, wherein the carbonate-bicarbonate buffer further comprises a nonionic detergent.

17. The method of claim 16, wherein the nonionic detergent is a polysorbate.

18. The method of claim 17, wherein the polysorbate is at a concentration in a range from 0.001% (v/v) to 1.0% (v/v).

19. The method of claim 14, wherein the mixture comprises the amount of animal feed and the carbonate-bicarbonate buffer at a ratio of less or equal to one selected from the group consisting of: 1:5 (w/v), 1:10 (w/v), 1:20 (w/v), 1:50 (w/v), 1:60 (w/v), 1:70 (w/v), 1:80 (w/v), 1:90 (w/v), 1:100 (w/v), 1:200 (w/v), 1:300 (w/v), 1:400 (w/v), 1:500 (w/v), 1:600 (w/v), 1:700 (w/v), 1:800 (w/v), 1:900 (w/v) and 1:1000 (w/v).

20. The method of claim 14, wherein the amount of animal feed is within a range from 100 g to 500 g.

21. The method of claim 15 further comprising grinding the animal feed to form flour, wherein the flour comprises particles having a size within a range of 250 μm to 6,000 μm.

22. The method of claim 21, wherein the size is at least 250 μm.

23. The method of claim 14, wherein a temperature of the mixture is in a range 20° C.-80° C.

24. The method of claim 14, wherein the temperature is 50° C.

25. The method of claim 14, further comprising measuring the activity of the feed enzyme.

26. The method of claim 14, wherein the feed enzyme is produced in a genetically engineered host.

27. The method of claim 26, wherein the host is selected from a plant cell, a bacterial cell, a mammalian cell, and a yeast cell.

28. The method of claim 14, wherein the feed enzyme is selected from the group comprising phytase, xylanase, glucanase, endoglucanase, cellobiohydrolase, amylase, protease, mannanase, arabinofuranosidase, xylosidase, glucoamylase, pectinase, lignin peroxidase, esterase, or cellulase.

29. The method of claim 28, wherein the phytase is an E. coli phytase.

30. The method of claim 25, wherein the step measuring is performed at a temperature within a range of 30° C.-80° C.

31. The method of claim 30, wherein the temperature is 37° C.
